(19) United States
(12) Patent Application Publication
(10) Pub. No.: US 2025/0353805 A1
(43) Pub. Date: Nov. 20, 2025

(54) PROCESS

(71) Applicant: GIVAUDAN SA, VERNIER (CH)

(72) Inventors: Fridtjof SCHROEDER, Kemptthal (CH); Martin LOVCHIK, Kemptthal (CH)

(21) Appl. No.: 18/716,728

(22) PCT Filed: Dec. 20, 2022
(86) PCT No.: PCT/EP2022/086858

§ 371 Date: Jun. 5, 2024

(30) Foreign Application Priority Data
Dec. 22, 2021 (GB) 2118717.4

(51) Int. Cl. C07C 17/383; C07C 7/04

(52) U.S. Cl. CPC C07C 17/383; C07C 7/04

(57) ABSTRACT

Described herein are methods for making intermediates useful in the production of fragrance ingredients starting from myrcene. In particular, methods for making geranyl chloride, (E)-6,10-dimethylundeca-1,5,9-triene and (E)-6,10-dimethylundeca-5,9-dien-1-yne are described. Methods for making other intermediates in the process are also described.

### TECHNICAL FIELD

The present invention relates to methods for making intermediates useful in the production of fragrances such as 2-oxygenated decalins (e.g. as described in WO 2020/173977) starting from biorenewable sources such as Myrcene. In particular, the invention relates to the production of a purified form of geranyl chloride.

### BACKGROUND

Monoterpenes, and particularly those derived from geraniol, are important building blocks for numerous fragrance compounds especially when synthesised from biorenewable sources. One synthetic method with industrial use includes amination of Myrcene and chlorination of the resulting Geranyl amine with a chloroformate to produce geranyl chloride, as described by Tanaka et al., (ChemLett, pp.1025-1026, 1977). Geranyl chloride is useful in the production of fragrance compounds, such as 2-oxygenated decalins (e.g. as described in WO 2020/173977, see in particular the passage bridging pages 5 and 6).

### SUMMARY

It has been found that a by-product of the chlorination of geranyl amine using a chloroformate is a carbamate. In any subsequent reaction of the geranyl chloride with a nucleophile, yield of the reaction can be reduced by side reactions of the carbamate with the nucleophile(s) and reaction of the thus-formed by-products with geranyl chloride.

It is therefore desirable to provide a new process by which geranyl chloride derived monoterpenes can be synthesised from biorenewable sources. Such a new process can improve the overall production of certain fragrances, such as 2-oxygenated decalins (e.g. as described in WO 2020/173977).

At its most general, the present invention provides a method of producing a purified form of geranyl chloride, wherein the method includes the step of distilling a reaction mixture comprising geranyl chloride and a carbamate in order to reduce the concentration of carbamate in at least one fraction containing geranyl chloride as compared to the undistilled reaction mixture.

In accordance with a first aspect of the present invention, there is provided a method of producing a purified form of the compound of Formula (I),

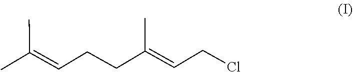

wherein the method comprises:
(i) a halogenation step comprising chlorinating a compound of Formula (II)

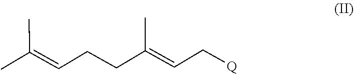

with
a) a chloroformate to produce a first reaction mixture comprising a compound of Formula (I) and a carbamate, or
b) an acid chloride to produce a first reaction mixture comprising a compound of Formula (I) and an amide,
   and
   (ii) a distillation step, the distillation step comprising distilling a mixture comprising the first reaction mixture to produce a separated portion of the distillation mixture comprising a purified form of the compound of Formula (I) as compared to the first reaction mixture; and
   wherein Q is a monovalent residue selected from N(methyl)₂, N(ethyl)₂, N(n-propyl)₂, N(isopropyl)₂, N(benzyl)₂, N(CH₃)CH₂CH₂NR²(CH₃), N-methyl-N-ethyl-amine,

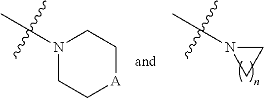

and wherein
n is selected from 1, 3 and 4; and
A is selected from O and NR² wherein R² is selected from H and geranyl.

In a second aspect of the present invention, there is provided a method of producing a compound of Formula (IV),

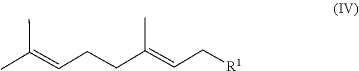

wherein the method comprises:
(i) a halogenation step comprising chlorinating a compound of Formula (II) with
   a) a chloroformate to produce a first reaction mixture comprising a compound of Formula (I) and a carbamate, or
   b) an acid chloride to produce a first reaction mixture comprising a compound of Formula (I) and an amide

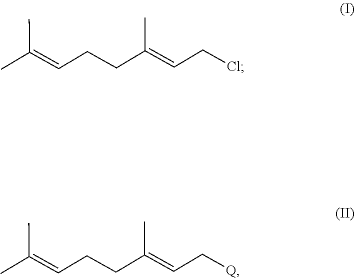

and
(ii) a distillation step, the distillation step comprising distilling a mixture comprising the first reaction mixture to produce a separated portion of the distillation mixture comprising a purified form of the compound of Formula (I) as compared to the first reaction mixture; and
(iii) contacting the separated portion with a nucleophile to produce a compound of formula (IV)

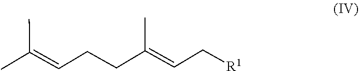

wherein
R¹ is selected from furyl, phenyl, C₁-C₁₀ alkyl, C₂-C₁₀ alkyl comprising at least one double bond or triplet bond, C₁-C₁₀ alkyl-OH, C₂-C₁₀ alkyl-OH comprising at least one double bond or one triplet bond (e.g. R¹ is prop-1-ene-3-yl, prop-1-yne-3-yl, 3-methylenebut-1-yne-4-yl, but-2-ene-4-yl, but-1-ene-3-yl, 3-methylbut-1,3-diene-4-yl, 3-methylenebutan-1-ol-4-yl, prop2-yne-3-yl or 2-methylenepropan-1-ol-3-yl), and —CH₂—C≡C—Si—(R³)₃ wherein each R³ is selected independently from C₁-C₅ alkyl (e.g. R¹ is 1-trimethylsilyl-prop-1-yne-3-yl); and Q has the same meaning as provided for the first aspect.

In a third aspect of the present invention, there is provided a method of producing a compound of Formula (IV),

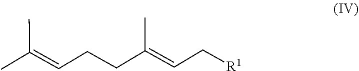

wherein the method comprises contacting the purified form of compound of Formula (I) obtained or obtainable by a method according to the first aspect with a nucleophile to produce a compound of Formula (IV), wherein R¹ has the same meaning as provided for the second aspect.

In a fourth aspect of the present invention, there is provided a purified form of compound of Formula (I),

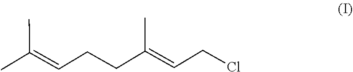

obtained by or obtainable by a method according to the first aspect.

In a fifth aspect of the present invention, there is provided a compound of Formula (IV),

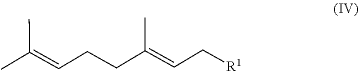

obtained or obtainable by a method according to the second or third aspects, wherein R¹ has the same meaning as provided for the second aspect.

The details, examples and preferences provided in relation to any particular one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

### FIGURES

FIG. 1: shows the synthetic route of the present invention

### DETAILED DESCRIPTION

The present invention provides novel and surprising methods for the production of a purified form of geranyl chloride (i.e. a compound of Formula (I)). The purified form of a compound of Formula (I) may be used in an improved production of compounds of Formula (IV), which in turn may be used to produce fragrances such as 2-oxygenated decalins (e.g. as described in WO 2020/173977).

In particular, the present invention is based, at least in part, on the surprising finding that distillation of a first reaction mixture comprising a compound of Formula (I) and a carbamate can provide a separated portion of the distillation mixture comprising a purified form of the compound of Formula (I) as compared to the first reaction mixture, following halogenation of a compound of Formula (II). This is particularly surprising because the compounds of Formula (I) may be thermally unstable. The method disclosed herein further allows the separated portion of geranyl chloride to react with nucleophiles with a reduced chance of formation of by-products in order to produce intermediates useful in making fragrance compounds.

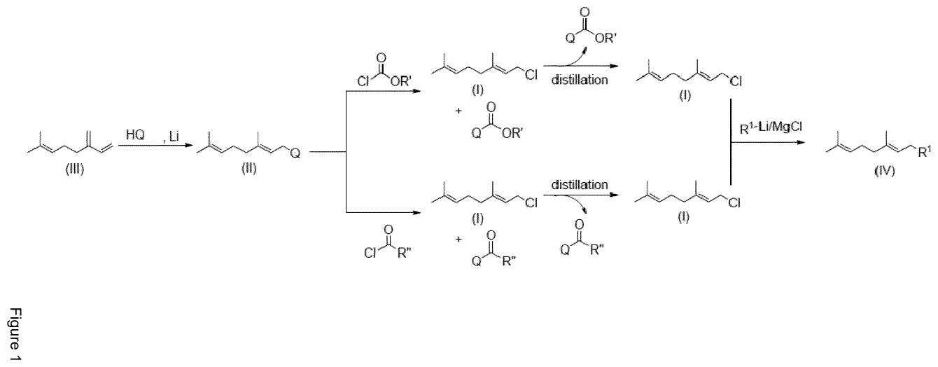

A synthetic route of the present invention is summarised in FIG. 1.

### Method of Producing a Purified Form of a Compound of Formula (I)

There is provided a method of producing a purified form of the compound of Formula (I),

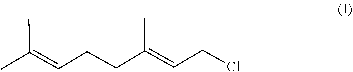

wherein the method comprises:
(i) a halogenation step comprising chlorinating a compound of Formula (II)

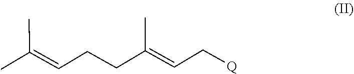

with
a) a chloroformate to produce a first reaction mixture comprising a compound of Formula (I) and a carbamate, or
b) an acid chloride to produce a first reaction mixture comprising a compound of Formula (I) and an amide,
   and
   (ii) a distillation step, the distillation step comprising distilling a mixture comprising the first reaction mixture to produce a separated portion of the distillation mixture comprising a purified form of the compound of Formula (I) as compared to the first reaction mixture; and
   wherein Q is a monovalent residue selected from N(methyl)₂, N(ethyl)₂, N(n-propyl)₂, N(isopropyl)₂, N(benzyl)₂, N(CH₃)CH₂CH₂NR²(CH₃), N-methyl-N-ethyl-amine,

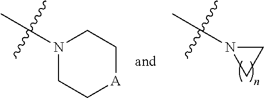

and wherein
n is selected from 1, 3 and 4; and
A is selected from O and NR² wherein R² is selected from H and geranyl.

### Formula (II)

The compound of Formula (II) is a geranyl amine. Q is a monovalent residue selected from N(methyl)₂, N(ethyl)₂, N(n-propyl)₂, N(isopropyl)₂, N(benzyl)₂, N(CH₃)CH₂CH₂NR²(CH₃), N-methyl-N-ethyl-amine, 25

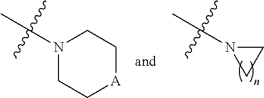

and wherein
n is selected from 1, 3 and 4; and
A is selected from O and NR² wherein R² is selected from H and geranyl.

Specific examples of compounds of Formula (II) include (E)-N,N-diethyl-3,7-dimethylocta2,6-dien-1-amine, or (E)-N,N-dipropyl-3,7-dimethylocta-2,6-dien-1-amine. The compound of Formula (II) may be the compound with CAS number [40267-53-6].

### Halogenation Step

The halogenation step comprises halogenating a compound of Formula (II) with a chlorformate to produce a first reaction mixture comprising a compound of Formula (I) and a carbamate. The chloroformate may be any suitable chloroformate of the Formula (VI):

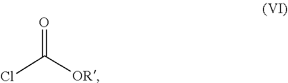

wherein R′ is selected from C₁-C₆ alkyl groups which may be optionally substituted.

In some embodiments, R′ is methyl, ethyl, n-propyl, isopropyl, n-butyl, sec-butyl, isobutyl, tert-butyl, n-pentyl, tert-pentyl, neopentyl, isopentyl, sec-pentyl, 3-pentyl, sec-isopentyl, or hexyl. In other embodiments, R′ is substituted methyl, substituted ethyl, substituted n-propyl, substituted isopropyl, substituted n-butyl, substituted sec-butyl, substituted isobutyl, substituted tert-butyl, substituted n-pentyl, substituted tert-pentyl, substituted neopentyl, substituted isopentyl, substituted sec-pentyl, substituted 3-pentyl, substituted sec-isopentyl, or substituted hexyl. In some examples, R′ is substituted methyl. In some examples, R′ is methyl substituted with an aryl group. In some examples, R′is benzyl. In some examples, R′ is naphthyl methyl.

In some embodiments, R′ is selected such that the chloroformate comprises a formic group that produces the carbamate in the halogenation step with a higher boiling point than the geranyl chloride (the compound of Formula (I)).

In some embodiments, R′ is selected such that the chloroformate comprises a formic group that produces the carbamate in the halogenation step with a lower boiling point than the compound of Formula (I).

In some embodiments, the chloroformate is methyl chloroformate. In some embodiments, the chlorformate is benzyl chloroformate. In some embodiments, the chloroformate is naphthyl methyl chloroformate.

Alternatively, the halogenation step comprises halogenating a compound of Formula (II) with an acid chloride.

The acid chloride may be any suitable acid chloride of Formula (VII)

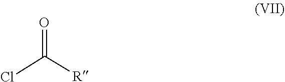

wherein R″ is selected from Cl, —C(O)Cl, phenyl wherein the phenyl ring is optionally substituted with one group selected from methyl, methoxy, halogen (e.g. Cl), C₁-C₁₀ alkyl (linear or branched) and —C(O)Cl, bicyclic aromatic systems and tricyclic aromatic systems (e.g. naphthyl). In some embodiments the acid chloride is oxalyl chloride.

In some embodiments, R″ is selected such that the acid chloride comprises an acid group that produces the amide (Q-C(O)-R″) in the halogenation step with a lower boiling point than the compound of Formula (I).

In some embodiments, R″ is selected such that the acid chloride comprises an acid group that produces an amide (Q-C(O)-R″) in the halogenation step with a higher boiling point than the compound of Formula (I). This is particularly the case when bisamides are formed from oxalyl chloride and terephthaloyl chloride, such as Q-C(O)-C(O)-Q and Q-C(O)-phenylene-C(O)-Q respectively.

In some embodiments the acid chloride is benzoyl chloride optionally substituted. In some embodiments the acid chloride is 4-methylbenzoyl chloride. In some embodiments the acid chloride is 4-methoxybenzoyl chloride. In some embodiments the acid chloride is p-chlorobenzoyl chloride. In some embodiments the acid chloride is terephthaloyl chloride.

Phosphoryl chloride is also suitable for the halogenation step giving the compound of Formula (I) and a hexaalkylphosphoramide (HAPA). However, due to the potentially toxicity of HAPA phosphoryl chloride is not an acceptable chlorination agent.

The halogenation step may be performed in an organic solvent or solvent-free. In particular embodiments, the halogenation step is performed in xylene.

### Formula (I)

The compound of Formula (I) is 1-chloro-3,7-dimethyl-2,6-octadiene enriched in the E-isomer (=geranyl chloride, (E)-geranyl chloride). In one particular embodiment the compound of Formula (I) has an enriched E/Z-isomer ratio of 95:5 to 99:1, or even higher.

### Distillation Step

The distillation step comprises distilling a mixture comprising the first reaction mixture to produce a separated portion of the mixture comprising a purified form of the compound of Formula (I) as compared to the first reaction mixture.

The first reaction mixture typically includes a compound of Formula (I), a carbamate or an amide and a solvent. The first reaction mixture may include other impurities. The purity of the compound of Formula (I) in the first reaction mixture depends on the other components in the first reaction mixture. The distillation step of the present invention separates out the components of the first reaction mixture to provide at least one separated portion of the first reaction mixture having the compound of Formula (I) at a higher purity level than in the first reaction mixture. In this way, the distillation step provides the compound of Formula (I) in a purified form as compared to the first reaction mixture.

As used herein, the term “a purified form of a compound of Formula (I)” or “compound of Formula (I) in a purified form” means that the “purified form” sample has a higher proportion of the compound of Formula (I) to other components in the sample as compared with the proportion of compound of Formula (I) to other components in the first reaction mixture. The basis on which to measure the proportion is not particularly limited as long as the measure is consistent as between the purified form and the first reaction mixture. For example, the proportion may be based on weight of the components in the purified form and the first reaction mixture. Alternatively, the ratio may be based on the molarity of the components in the purified form and the first reaction mixture. Various methods are known per se for determining the ratio of components in such mixtures. For example, GC with internal or external standard or GC-MS (e.g. area under a peak) and/or calibrated NMR (optionally using analysis software, such as qNMR) of the purified form and first reaction mixture may be used to determine the ratios. In some embodiments, the purity of the compound of Formula (I) may be expressed as a percentage relative to the total amount (e.g. by weight) of the sample. For example, the separated portion may contain 85% by weight of the compound of Formula (I) (with 15% by weight being other components) and may be described as having a purity of 85%.

In some embodiments, the purity of the compound of Formula (I) in the separated portion is 50% or more, 55% or more, 60% or more, 65% or more, or 70% or more as measured by NMR and analyses using qNMR. In some embodiments, the purity of the compound of Formula (I) in the separated portion is 60% or more, 65% or more, 70% or more, 75% or more, 80% or more, 85% or more, or 90% or more as measured by GC-MS.

The distillation step typically separates the mixture comprising the first reaction mixture into a distilled distillation mixture with one or more distillate fractions. The separated portion(s) having a higher purity of the compound of Formula (I) may be one or more distillate fractions. In other words, the separated portion having a higher purity of the compound of Formula (I) may be one or more fraction of the distillate. The separated portion having a higher purity of the compound of Formula (I) may be the distilled distillation mixture. Alternatively, the separated portion having a higher purity of the compound of Formula (I) may be the remaining portion of the distillation mixture after the distillates have been removed from the distillation mixture by the distillation step.

In some embodiments, the separated portion having a higher purity of the compound of Formula (I) is a single distillate fraction. In other embodiments, the separated portion having a higher purity of the compound of Formula (I) is solely the distilled distillation mixture. In particular embodiments, the separated portion may be a combination of one of more distillate fraction.

In particular embodiments, the separated portion has a higher molar or weight ratio of the compound of Formula (I) to carbamate or amide, respectively, than the molar or weight ratio, respectively, of the compound of Formula (I) to carbamate or amide, respectively, in the first reaction mixture. The distillation step may effectively separate at least some of the compound of Formula (I) from at least some of the carbamate or amide, respectively, produced in the halogenation step. In this way, the further reaction of compound of Formula (I) after the distillation step may proceed with fewer side-reactions caused by the presence of carbamate/amid, respectively.

In all aspects of the present invention, the distillation step may comprise distilling a distillation mixture comprising the first reaction mixture to produce a separated portion of the distillation mixture having a higher molar or weight ratio of the compound of Formula (I) to carbamate or amide, respectively, than the molar or weight ratio, respectively, of the compound of Formula (I) to carbamate or amide, respectively, in the first reaction mixture.

In some embodiments, the separated portion includes less than 5% by weight, less than 4% by weight, less than 3% by weight, less than 2% by weight, less than 1% by weight, less than 0.5% by weight, less than 0.1% by weight, less than 0.01% by weight of carbamate or amide, respectively. The weight percentage of carbamate or amide, respectively, may be measured using GCMS. In particular embodiments, the separated portion is substantially free of carbamate or amide, respectively.

In certain embodiments, the separated portion has more than 80% by weight of the compound of Formula (I) and less than 5% by weight of carbamate or amide, respectively, as measured by GCMS. In particular embodiments, the separated portion has more than more than 85% by weight of the compound of Formula (I) and less than 2% by weight of carbamate or amide, respectively, as measured by GCMS. In more particular embodiments, the separated portion has more than 90% by weight of the compound of Formula (I) and less than 1% by weight of carbamate or amide, respectively, as measured by GCMS.

The carbamate being separated from the compound of Formula (I) (or vice versa) will depend on the chloroformate used in the halogenation step. The carbamate has a general formula QC(═O)OR′, where Q and R′ are as defined herein.

In some embodiments, where the carbamate has a higher boiling point than the compound of Formula (I), the compound of Formula (I) is distilled from the carbamate. In these embodiments, the molecular weight of the carbamate is higher than the molecular weight of the compound of Formula (I). In these embodiments, the chloroformate is, for example, benzyl or naphthyl methyl chloroformate.

In some embodiments, where the carbamate has a lower boiling point than the compound of Formula (I), the carbamate is distilled from the compound of Formula (I). In these embodiments, the molecular weight of the carbamate is lower than the molecular weight of the compound of Formula (I). In these embodiments, the chloroformate may be, for example, methyl chloroformate.

The amide being separated from the compound of Formula (I) (or vice versa) will depend on the acid chloride used in the halogenation step. The amide has a general formula QC(═O)R″, where Q and R″ are as defined herein.

In some embodiments, where the amide has a higher boiling point than the compound of Formula (I), the compound of Formula (I) is distilled from the amide. In these embodiments the acid chloride may be relatively large to form an amide having a higher molecular weight than the compound of Formula (I), e.g. the compound of Formula (VII) may be benzoyl chloride or substituted benzoyl chlorides such as methyl-or methoxy-substituted benzoyl chloride or terephthaloyl chloride. With terephthaloyl chloride and oxalyl chloride bisamides can be formed having a higher molecular weight than the compound of Formula (I).

In some embodiments, where the amide has a lower boiling point than the compound of Formula (I), the amide is distilled from the compound of Formula (I). In these embodiments the acid chloride may be of low molecular weight such as formyl chloride, acetyl chloride or propionylchloride.

The distillation step may be performed at an elevated temperature. In other words, the distillation step may be performed at a temperature above ambient or room temperature. The distillation step may be performed at a temperature between the boiling point of the carbamate or amide, respectively, and the boiling point of the compound of Formula (I). The boiling point of each of the carbamate or amide, respectively, and the compound of Formula (I) will depend on the pressure at which the distillation is performed. The boiling points at various pressures will be known per se or can be determined by heating the sample at a given pressure.

The distillation step may be performed at a temperature of 60° C. or more, 65° C. or more, 75° C. or more, 80° C. or more, 85° C. or more, 90° C. or more, or 95° C. or more. In some embodiments, the distillation step is performed in the range of 60° C. to 105° C. In some embodiments, the distillation step is performed in the range of at 70° C. to 95° C. In alternative embodiments, the distillation step is performed in the range of 74° C. to 100° C.

In some embodiments, the distillation comprises separation under vacuum. Distillation devices may comprise short-path distillation, distillation through a fractionation column or thin-film distillation. The choice of the equipment depends on the relative boiling point of the compound of Formula (I) compared to the carbamate or amide, respectively.

In some embodiments, vacuum distillation comprises distillation at a reduced pressure of up to about 100 mbar, for example, up to about 75 mbar, up to about 60 mbar, up to about 50 mbar, up to about 45 mbar, up to about 40 mbar, up to about 35 mbar, up to about 30 mbar, up to about 25 mbar, up to about 20 mbar, up to about 15 mbar, up to about 10 mbar, up to about 5 mbar, up to about 2 mbar, up to about 1 mbar. In some examples, vacuum distillation comprises distillation at a reduced pressure of from about 1 mbar to about 100 mbar, for example, from about 2 mbar to about 75 mbar, about 5 mbar to about 60 mbar, about 10 mbar to about 50 mbar. In other embodiments, vacuum distillation comprises distillation at a reduced pressure of up to about 0.9 mbar, up to about 0.8 mbar, up to about 0.7 mbar, up to about 0.6 mbar, up to about 0.5 mbar, up to about 0.4 mbar, up to about 0.3 mbar, up to about 0.2 mbar, up to about 0.1 mbar. In some examples, the vacuum distillation comprises distillation at a reduced pressure in the range of about 0.001 mbar to about 0.9 mbar, of about 0.005 mbar to about 0.5 mbar, about 0.1 mbar to about 0.3 mbar.

In some embodiments, the distillation step is performed in the range of 60° C. to 105° C. and at a reduced pressure in the range of from about 0.1 mbar to about 100 mbar. In some embodiments, the distillation step is performed in the range of at 70° C. to 95° C. and at a reduced pressure in the range of about 1 mbar to about 60 mbar. In alternative embodiments, the distillation step is performed in the range of 74° C. to 100° C. and at a reduced pressure in the range of about 0.1 mbar to about 0.3 mbar.

The distillation step may comprise distillation at different temperatures and/or reduced pressures to provide more than one fraction. This is a known technique per se in distillation. For example, the distillation step may comprise distillation: (i) at a first temperature and first pressure to provide a first fraction; and (ii) at a second temperature and/or second pressure to provide a second fraction. In other words, one or both of the temperature and pressure of distillation may be varied to provide the second distillation. The temperature and/or pressure of the distillation may be further varied in order to provide further fractions as desired.

In addition to separate at least some of the compound of Formula (I) from at least some of the carbamate or amide, respectively, formed in the halogenation step the distillation step may further separate at least some of the compound of Formula (I) from other components in the first reaction mixture. For example, the distillation step may separate some or all of the solvent from the compound of Formula (I) in one or more fractions.

In some embodiments, a phase separation step is performed on the first reaction mixture before the distillation step. The halogenation step is typically performed in an organic solvent. In these embodiments, the phase separation step comprises adding an aqueous phase to the first reaction mixture and separating the organic phase from the aqueous phase. The compound of Formula (I) will typically remain in the organic phase. The distillation step may be performed on the organic phase resulting from the phase separation.

After phase separation and before the distillation step, the organic phase containing the compound of Formula (I) may be neutralised. Neutralisation may be performed by washing the organic phase with a basic aqueous solution, such as saturated aqueous solutions of sodium bicarbonate (NaHCO₃) or sodium carbonate (Na₂CO₃). Such neutralisation of organic phases are known per se and other neutralisation techniques may be used.

### Amination Step

The halogenation step is preceded by an amination step comprising aminating myrcene (7-methyl-3-methylene-1,6-octadiene) a compound of Formula (III),

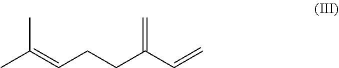

to produce the compound of Formula (II).

Myrcene may, for example, be obtained commercially and is usually produced from beta-Pinene through pyrolysis. Alternatively, myrcene may be synthesised by carbon-carbon bond formation between two isoprene units or from geraniol.

The amination step comprises aminating a compound of Formula (III) to produce the compound of Formula (II). In some embodiments, aminating a compound of Formula (III) comprises contacting a compound of Formula (III) with an amine in the presence of a catalyst.

The amine is a secondary amine with a formula of HQ where Q is as defined herein. In some embodiments, the catalyst is Lithium in other embodiments sodium or alkyl lithium, especially butyl lithium.

### Methods of Making a Compound of Formula (IV)

In an aspect, there is provided a method for making a compound of Formula (IV),

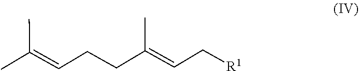

wherein the method comprises contacting the compound of Formula (I),

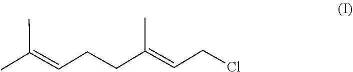

with a nucleophile, wherein R¹ is selected from furyl, phenyl, C₁-C₁₀ alkyl, C₂-C₁₀ alkyl comprising at least one double bond or triplet bond, C₁-C₁₀ alkyl-OH, C₂-C₁₀ alkyl-OH comprising at least one double bond or one triplet bond (e.g. R¹ is prop-1-ene-3-yl, prop-1-yne-3-yl, 3-methylenebut-1-yne-4-yl, but-2-ene-4-yl, but-1-ene-3-yl, 3-methylbut-1,3-diene-4-yl, 3-methylenebutan-1-ol-4-yl, prop-2-yne-3-yl or 2-methylenepropan-1-ol-3-yl), and —CH₂-C≡C-Si-(R³)₃ wherein each R³ is selected independently from C₁-C₅ alkyl (e.g. R¹ is 1-trimethylsilyl-prop-1- yne-3-yl).

The compound of Formula (I) may either be obtained or obtainable by a method according to the first aspect, or the method includes the steps described herein for making the compound of Formula (I).

### Formula (IV)

The compound of Formula (IV) is derived from geranyl chloride (I).

In a specific embodiment, the compound of Formula (IV) is (E)-6,10-dimethylundeca-5,9-dien-1-yne. The compound of Formula (IV) may be the compound with CAS number[100451-98-7].

In another embodiment, the compound of Formula (IV) is (E)-6,10-dimethylundeca-1,5,9-triene. The compound of Formula (IV) may be the compound with CAS number [24120-53-4].

### Nucleophile

The method for making a compound of Formula (IV) comprises reacting the compound of Formula (I) with a nucleophile.

In particular embodiments, the nucleophile is an organometallic reagent, for example, an allyl magnesium halide reagent or an alkynyllithium reagent. For example, the nucleophile may be an alkyne (e.g. a terminal alkyne, such as propyne) lithiated with an alkyllithium (such as hexyl lithium). Specific examples of nucleophiles include, but are not limited to, allyl magnesium chloride and propyne lithiated with hexyl lithium or butyl lithium.

In some embodiments, the reaction is as described in the passage bridging pages 5 and 6 of WO 2020/173977, the contents of which is incorporated herein.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. The term “comprising” also means “including” as well as “consisting” e.g. a composition “comprising” X may consist exclusively of X or may include something additional e.g. X+Y. It must be noted also that, as used in this specification and the appended claims, the singular forms “a”, “an” and “the” include plural referents unless the content clearly dictates otherwise. By way of example, a reference to “an enzyme” is a reference to “one or more enzymes”.

It is to be understood that this disclosure is not limited to the particular methodology, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by the person skilled in the art. In accordance with the present disclosure there may be conventional techniques employed which are within the skill of the art.

This disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting.

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety.

As used herein, “alkyl” may refer to a linear straight chain or branched-chain saturated hydrocarbon group, unless specified otherwise. The alkyl may, for example, unless specified otherwise, have from 1 to 6 carbon atoms, for example from 1 to 5 carbon atoms, for example from 1 to 4 carbon atoms, for example from 1 to 3 carbon atoms, for example 1 or 2 carbon atoms.

As used herein, “alkyl comprising at least one double or triple bond” may refer to linear straight chain or branched-chain unsaturated hydrocarbon group. The chain may, for example, unless specified otherwise, comprise 1-5 double bonds, e.g., 1-4, 1-3, 1-2, which include both endo-and/or exo-double bounds; or the chain may, for example, unless specified otherwise, comprise 1-3, e.g., 2, triple bonds, which include both endo-and/or exotriple bonds; or the chain may, for example, unless specified otherwise, comprise 1-5 double bonds (e.g. 2, 3, 4 double bonds) and 1-3 triple bonds (e.g., 2 triple bonds), which include both endo-and/or exo-bonds.

As used herein, “bicyclic aromatic systems” refers to hydrocarbon residues comprising two joined aromatic rings. Optionally, one or both of the aromatic rings may be substituted with one or two groups selected from methyl and methoxy and/or one —C(O)CI group. The bicyclic aromatic system, is, for example, 1-napthyl, 2-naphthyl, 4-methyl-1-naphthyl, 2-ethoxy-1-naphthyl, 6-methoxy-2-naphthyl, 6-methoxy-2-naphthyl, and 2-naphthoyl chloride-6-yl.

As used herein, “tricyclic aromatic systems” refers to hydrocarbon residues comprising three joined aromatic rings. The tricyclic aromatic system is, for example, 9-anthracenyl.

The examples described herein are illustrative of the present disclosure and are not intended to be limitations thereon. Different embodiments of the present disclosure have been described according to the present disclosure. Many modifications and variations may be made to the techniques described and illustrated herein without departing from the spirit and scope of the disclosure. Accordingly, it should be understood that the examples are illustrative only and are not limiting upon the scope of the disclosure.

### EXAMPLES

The following illustrates examples of the methods and other aspects described herein. Thus, these Examples should not be considered as limitations of the present disclosure but are merely in place to teach how to make examples of the present disclosure.

### Example 1: Synthesis of Pure Geranyl Chloride from Myrcene Through Distillative Removal of Methyl Diethyl Carbamate

Lithium (1.3 g, 0.19 mol) and styrene (24 g, 0.23 mol) were added to myrcene 95% (600 g, 4.2 mol) and diethylamine (323 g, 4.4 mol) at 60° C. under nitrogen and stirring. After 40 min the lithium metal was dissolved almost completely and after 2 h full conversion to the (E)-N,N-diethyl-3,7-dimethylocta-2,6-dien-1-amine intermediate was detected by GC. At 10-15° C. methyl chloroformate (439 g, 4.6 mol) in xylene (352 g) was added dropwise over an hour. After complete addition, the reaction mass was stirred for another 15 min at 25° C. Water (500 g) was added, the layers were separated, and the organic phase washed with sat. NaHCO₃ (250 g). From the organic phase (1531 g) xylene and the methyl diethyl carbamate byproduct were removed by distillation at 90° C. (50-15 mbar) through a distillation bridge, giving 937 g of volatiles and crude geranyl chloride (545 g) which was used for derivatisation (e.g. propargylation in Example 2) without further purification.

TABLE 1
Distillation

| | | | | | methyl | |
|---|---|---|---|---|---|---|
| | oil bath | pressure | fraction | | diethyl- | geranyl |
| Fraction | [° C.] | [mbar] | [g] | xylene ^{a} | carbamate ^{a} | chloride ^{a} |
| 1 | 90 | 50 | 450.3 | 71 | 21 | 1 |
| 2 | 90 | 33 | 459.8 | 36 | 43 | 6 |
| 3 | 95 | 15 | 27.3 | 2 | 35 | 13 |
| Pot | | | 545.0 | 0 | 0 | 74 |
| Total | | | 1482 ^{b} | | | |

Distillation of the carbamate/xylene mixture from the organic phase (1531 g) after aqueous work-up. a) GC rpa in %. b) Large quantities of volatiles were found in the cooling trap which explains the discrepancy (49 g) of the organic material before and after distillation.

Molar yield: 56% based on myrcene and corrected by qNMR. Purity: 74% (qNMR), 95% (GCMS). EZ-ratio: 99:1 (GCMS). The analytical data of geranyl chloride were identical with the ones obtained for this compound prepared as described in the literature.

### Example 2: Synthesis of (E)-6,10-dimethylundeca-5,9-dien-1-yne from Pure Propyne and Pure Geranyl Chloride Generated from Myrcene

Propyne (45 g, 1.125 mol), condensed at −40° C. into a flask equipped with −78° C. cooling finger, was added over 1.5 h via double needle into absolute THF (859 g), precooled to −20° C. under nitrogen, followed by 2.4 M hexyl lithium in hexane (450 ml, 1.125 mol) at −20° C. under stirring. The cooling bath was removed and a second portion of 2.5 M hexyl lithium (450 ml, 1.125 mol) added over 30 min at 25° C. After stirring for 30 min the suspension was cooled to −15° C., and geranyl chloride (178 g, 765 mmol, prepared and distilled as in example 1 with 77% purity, q-NMR) was added dropwise over 1 h at −10° C. After 45 min, another portion of geranyl chloride (17 g, 73 mmol) was added, followed by a last portion of geranyl chloride (17 g, 73 mmol) after another 45 min. After 2 h at −10° C., the orange reaction mass was quenched with sat. NH₄Cl (1 litre) followed by water (1 litre) under cooling. After phase separation, the aqueous layer was extracted with hexane. The combined organic phases were washed with water (1 litre) and brine (1 litre). After drying over MgSO₄, the solvents were removed under reduced pressure (45° C., 6 mbar) giving 214 g of crude title compound with 67% purity (qNMR). After addition of paraffine oil (24 g), tridodecylamine (2.2 g) and tocopherol (0.3 g), the crude was distilled at 60° C./0.03 mbar giving 147 g distillate in 6 fractions. Fractions 2-5 were pooled giving 139 g of the title compound. Molar yield: 90% (crude, corr, based on geranyl chloride), 50% (crude, corr, based on myrcene), 79% (pooled, based on geranyl chloride). Purity: 94% (pooled fractions, GCMS). EZ-ratio: 98:2 (GCMS). The analytical data of (E)-6, 10-dimethylundeca-5,9-dien-1-yne were identical with the ones obtained for this compound prepared as described in the literature.

### Example 3: Synthesis of (E)-6,10-dimethylundeca-1,5,9-triene from Allylmagnesium Chloride and Pure Geranyl Chloride Generated from Myrcene

Lithium (0.25 g, 36 mmol) and styrene (4.6 g, 44 mmol) were added to myrcene 95% (115 g, 0.8 mol) and diethylamine (61.7 g, 0.84 mol) at 60° C. under nitrogen and stirring. After 40 min, the lithium metal was dissolved almost completely and after 2 h full conversion to the (E)-N,N-diethyl-3,7-dimethylocta-2,6-dien-1-amine intermediate was detected by GC. At 10-15° C., methyl chloroformate (84 g, 0.88 mol) in xylene (69 g) was added dropwise over an hour. After complete addition, the reaction mass was stirred for another 30 min at 25° C. Water (150 g) was added, the layers were separated, and the organic phase washed with sat. NaHCO3 (100 g). From the organic phase, xylene and the methyl diethyl carbamate byproduct were removed by distillation at 70-90° C. (50-20 mbar) through a distillation bridge, giving 166 g of volatiles. The remaining crude geranyl chloride was mixed with absolute THF, and allylmagnesium chloride (1.7 M in THF, 520 ml, 0.88 mol) was added dropwise over 50 min at 0-10° C. After complete addition, the reaction mass was stirred for another 15 min. Complete conversion of the geranyl chloride was checked by GC. The reaction mass was cooled to 10° C., and 10% aqueous acetic acid (300 g) was slowly added under stirring, followed by tert-butyl methyl ether (150 g). The aqueous phase was separated and the organic layer washed with water (250 g) and brine (100 g). The organic layer was dried over MgSO₄, filtered and evaporated under reduced pressure giving the title compound as yellowish oil which was fractionated at 40-45° C./0.05-0.04 mbar, giving 79.4 g pooled fractions of (E)-6,10-dimethylundeca-1,5,9-triene. Molar yield: 54% based on myrcene and corrected by purity. Purity: 98% (GCMS). EZ-ratio: 99:1 (GCMS). The analytical data of (E)-6, 10-dimethylundeca-1,5,9-triene were identical with the ones from the literature.

### Example 4: Synthesis of Pure Geranyl Chloride from Myrcene Through Distillation from Benzyl Dipropylcarbamate

Lithium (0.35 g, 50 mmol) and styrene (6.4 g, 61 mmol) were added to myrcene 88% (152 g, 1.04 mol) and dipropylamine (113 g, 1.1 mol) at 65° C. under nitrogen and stirring. After 40 min, the lithium metal was dissolved almost completely and after 22 h, a 98% conversion to (E)-3,7-dimethyl-N,N-dipropylocta-2,6-dien-1-amine was detected by GC. At 5° C., benzyl chloroformate (171 g, 0.975 mol) was added dropwise over 40 min. After complete addition, the reaction mass was stirred for another 25 min at 25° C., then another portion of benzyl chloroformate (9.5 g, 54 mmol) was added. After 30 min, a 99% conversion of (E)-3,7-dimethyl-N,N-dipropylocta-2,6-dien-1-amine was detected by GC. Geranyl chloride was distilled from this mixture (447 g) at 74-100° C./0.07-0.03 mbar giving 175 g of a distillate. Fractions 3-6 were pooled giving geranyl chloride with 85% purity.

TABLE 2

| | T bath | T head | p | fraction | % BnCl | % Ge—Cl | % Ge-A | % carbamate |
|---|---|---|---|---|---|---|---|---|
| Fraction | [° C.] | [° C.] | [mbar] | [g] | t_{R} 0.88 | t_{R} 2.36 | t_{R} 5.31 | t_{R} 6.39 |
| 1 | 75 | 25-41 | 0.07 | 8.2 | 17 | 14.7 | | |
| 2 | 75 | 42-48 | 0.05 | 25.5 | 15 | 41.3 | | |
| 3 | 75 | 49 | 0.05 | 22.7 | 12 | 64.3 | | |
| 4 | 85 | 49-51 | 0.05 | 29.5 | 7 | 80.4 | | |
| 5 | 95 | 52-54 | 0.07 | 76.0 | 1 | 92.1 | | |
| 6 | 100 | 53 | 0.03 | 7.4 | | 89.2 | | |
| 7 | 100 | 53-78 | 0.03 | 5.6 | | 30.3 | 5 | |
| residue | | | | 265.2 | | 0.4 | 6 | 85 |
| total in g | dist: 175 g | | | | 10.8 g | 129.4 g | | 225.4 g |
| (mol %) | | | | | (8%) ^{a} | (72%) ^{a} | | (94%) ^{b} |

Crude geranyl chloride was distilled through a 10 cm vigreux column. Fractions F3-6 were pooled giving 136 g of pure product. T=temperature, p=pressure. BnCI=benzyl chloride [M 126.6]. Ge-CI=geranyl chloride. Ge-A=(E)-3,7-dimethyl-N,N-dipropylocta-2,6-dien-1-amine [M 237]. Carbamate=benzyl dipropylcarbamate [M 235.2]. t_{R}=GC retention time in minutes. a) molar yield based on Myrcene. b) molar yield based on benzyl chloroformate. c) carbamate=benzyl dipropylcarbamate.

Molar yield: 76% based on myrcene 88% (all fractions), 68% based on myrcene 88% (pooled fractions 3-6). Purity: 85% (qNMR), 95% (GCMS). EZ-ratio: >98:2 (GCMS). Impurities: benzyl chloride (3%). The analytical data of geranyl chloride were identical with the ones obtained for this compound prepared as described in the literature.

Methyl chloroformate was replaced by benzyl chloroformate; this allows a quicker distillation (compared to Example 1). Instead of Ge-Cl remaining in the flask, Ge-Cl is the compound which is distilled from solution. Instead of benzyl chloroformate one may use naphthyl methyl chloroformate.

### Example 5: Synthesis of (E)-6,10-dimethylundeca-1,5,9-triene from Allylmagnesium Chloride and Pure Geranyl Chloride Obtained by Distillation

Under nitrogen, magnesium powder (22.2 g, 0.83 mol) was suspended in 2-methyltetrahydrofuran (1.1 litre) and activated with an iodine particle. By dropwise addition of allyl chloride (66 ml, 0.8 mol) the temperature of the reaction mixture is slowly increased and kept at reflux during addition (1.5 h) and another 0.5 h. Then, geranyl chloride (from example 5) was added dropwise at reflux over 3 h and stirred another 1 h at reflux. After cooling to 10° C., the grey reaction mixture was quenched by slow addition of 2 M HCl (0.8 litre). The yellowish clear biphasic mixture was separated and the organic layer washed with 2 M NaOH (0.6 litre), dried over MgSO₄, filtered and evaporated giving 138 g crude product as a yellow liquid. Distillation gave 114 g of a distillate from which fractions 3 and 4 were pooled giving 84.5 g of (E)-6, 10-dimethylundeca-1,5,9-triene. Molar yield: 81% (all fractions, corr, based on geranyl chloride), 67% (pooled fractions, based on geranyl chloride), 61% (pooled fractions based on myrcene). Purity: 94% (pooled fractions, GCMS). E/Z-ratio: 96:4

(GCMS). Impurities: 4,8-dimethyl-4-vinylnona-1,7-diene (2%), (Z)-6,10-dimethylundeca1,5,9-triene (4%). The analytical data of triene were identical with the ones from the literature.

### Comparative Example 1: Synthesis of (E)-6,10-dimethylundeca-1,5,9-triene from Allylmagnesium Chloride and a Mixture of Geranyl Chloride in Low Boiling Solvents without Distillative Removal of the Carbamate

Instead of xylene, lower boiling solvents such as tert-butyl methyl ether and hexane were used in this experiment for better evaporation of these solvents from the E-geranyl chloride/methyl diethyl carbamate mixture.

Butyl lithium 1.6 M in hexane (106 ml, 173 mmol) was added dropwise within 20 min to myrcene 95% (750 g, 5.23 mol) and diethylamine (405 g, 5.5 mol) at 60° C. under nitrogen and stirring. After 2 h stirring at this temperature and 98% conversion to the (E)-N, N-diethyl-3,7-dimethylocta-2,6-dien-1-amineintermediate, methyl chloroformate (578 g, 6.1 mol) was added at −5° C. The viscous reaction mass was stirred for another 30 min, with full conversion of geranyl amine to a mixture of methyl diethyl carbamate and geranyl chloride detected by GC. Water (1 litre) was added, followed by extraction with tert-butyl methyl ether. The organic phase was washed with sat. NaHCO₃ and brine, dried over MgSO₄ and evaporated. The crude residue (1160 g) contained methyl diethyl carbamate (44% rpa) and geranyl chloride (41% rpa) according to GC.

5 g of this mixture (containing ˜12 mmol geranyl chloride and methyl diethyl carbamate) were dissolved in absolute THF (45 ml) under nitrogen and stirring. Allylmagnesium chloride 1.45 M in THF was added dropwise in three portions (3×5.4 ml, 23 mmol) at −10° C. After 19 h stirring at 25° C., the reaction mass was quenched with sat. NH₄Cl (70 ml). After phase separation and extraction with tert-butyl methyl ether the organic phases were combined, dried over MgSO₄, filtered and evaporated giving 4.6 g of a red residue containing methyl diethyl carbamate (17% rpa), geranyl chloride (20% rpa), geranyl diethyl amine (16%) and only traces of (E)-6, 10-dimethylundeca-1,5,9-triene.

The re-formation of geranyl diethyl amine, which had been completely converted to geranyl chloride, can be only explained by allylation of the methyl diethyl carbamate impurity, thus liberating Et₂N-MgCl which reacts with geranyl chloride. Thus, the presence of the carbamate is detrimental to any organometallic derivatization of geranyl chloride.

The analytical data of E-geranyl diethyl amine are consistent with the ones from the literature.

### Comparative Example 2: Synthesis of (E)-6,10-dimethylundeca-1,5,9-triene from Allylmagnesium Chloride and a Mixture of Geranyl Chloride in Xylene without Distillative Removal of the Carbamate

As described in Example 1 a geranyl chloride/methyl diethyl carbamate mixture in xylene was prepared from Lithium (0.76 g, 0.11 mol), styrene (13.8 g, 0.13 mol), myrcene 95% (345 g, 2.41 mol) and diethylamine (185 g, 2.53 mol) followed by work-up as described but only partial removal of xylene giving a yellow liquid (768 g) consisting of xylene (20%), methyl diethyl carbamate (26%), geranyl chloride (37%) and geranyl diethyl amine according to GCMS, containing ca. 1.3 mol of Geranyl chloride (estimated according to Example 1).

5 g of this liquid (16 mmol geranyl Chloride) were dissolved in water-free THF (45 ml) and allylmagnesium chloride 2 M in THF was added dropwise in three portions (3×2.9 ml, 17 mmol) at −10° C. After 16 h stirring at 25° C., the reaction mass was quenched with sat. NH₄Cl (50 ml). After phase separation and extraction with tert-butyl methyl ether the organic phases were combined, dried over MgSO₄, filtered and evaporated giving 4.1 g of a red residue containing xylene (14%), methyl diethyl carbamate (13% rpa), geranyl chloride (31% rpa), geranyl diethyl amine (9%). (E)-6,10-Dimethylundeca-1,5,9-triene was not detected. For consistent analytical data and a conclusion see comparison Example 1.

While the invention has been described with reference to certain examples, those skilled in the art will appreciate that various modifications, changes, omissions, and substitutions can be made without departing from the spirit of the disclosure. It is intended, therefore, that the invention be limited by the scope of the following claims and their equivalents. Unless otherwise stated, the features of any dependent claim can be combined with the features of any of the other dependent claims and any of the independent claims.

1. A method producing a purified form of geranyl chloride, a compound of Formula (I), 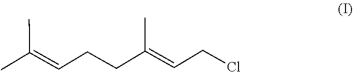
wherein the method comprises:
(i) a halogenation step comprising chlorinating a compound of Formula (II) 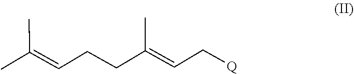
with
a) a chloroformate to produce a first reaction mixture comprising a compound of Formula (I) and a carbamate, or
b) an acid chloride to produce a first reaction mixture comprising a compound of Formula (I) and an amide,
and
(ii) a distillation step, the distillation step comprising distilling a mixture comprising the first reaction mixture to produce a separated portion of the distillation mixture comprising a purified form of the compound of Formula (I) as compared to the first reaction mixture; and
wherein Q is a a monovalent residue selected from N(methyl)₂, N(ethyl)₂, N(n-propyl)₂, N(isopropyl)₂, N(benzyl)₂, N(CH₃)CH₂CH₂NR²(CH₃), N-methyl-N-ethyl-amine, 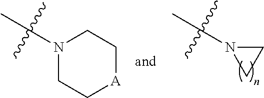
and wherein
n is selected from 1, 3 and 4; and
A is selected from O and NR² wherein R² is selected from H and geranyl

2. The method of claim 1 wherein the chloroformate is a compound of the Formula (VI), 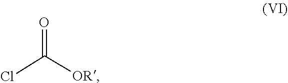
wherein R′ is selected from C₁-C₆ alkyl groups which may be optionally substituted, such that the carbamate has the formula Q-C(═O)OR′.

3. The method of claim 1 wherein the acid chloride is a compound of Formula (VII), 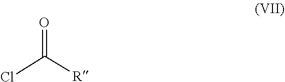
wherein R″ is selected from selected from Cl, —C(O)Cl, phenyl wherein the phenyl ring is optionally substituted with one group selected from methyl, methoxy, halogen, C₁-C₁₀ alkyl (linear or branched) and —C(O)Cl, bicyclic aromatic systems and tricyclic aromatic systems, such sat the amide has the formula Q-C(═O)R″.

4. The method of any one of claims 1 to 2 wherein carbamate has a higher boiling point than the compound of Formula (I) and the compound of Formula (I) is distilled from distillation mixture into one or more distillate fractions.

5. The method of any one of claims 1 to 2 wherein the carbamate has a lower boiling point than the compound of Formula (I) and the carbamate is distilled from the distillation mixture into one or more distillate fractions.

6. The method of any preceding claim wherein the separated portion has a higher molar or weight ratio of the compound of Formula (I) to carbamate than the molar or weight ratio, respectively, of the compound of Formula (I) to carbamate in the first reaction mixture.

7. A method of producing a compound of Formula (IV), 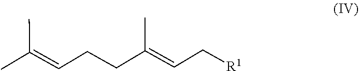
wherein the method comprises:
(i) a halogenation step comprising chlorination of a compound of Formula (II) with
a) a chloroformate to produce a first reaction mixture comprising a compound of Formula (I) and a carbamate, or
b) an acid chloride to produce a first reaction mixture comprising a compound of Formula (I) and an amide 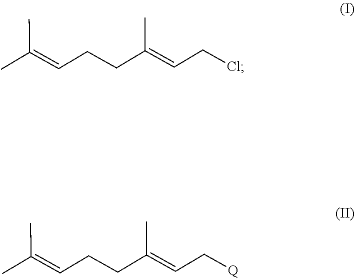
wherein Q is a a monovalent residue selected from N(methyl)₂, N(ethyl)₂, N(n-propyl)₂, N(isopropyl)₂, N(benzyl)₂, N(CH₃)CH₂CH₂NR²(CH₃), N-methyl-N-ethyl-amine, 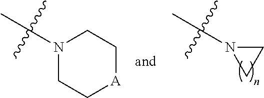
and wherein
n is selected from 1, 3 and 4; and
A is selected from O and NR² wherein R² is selected from H and geranyl;
and
(ii) a distillation step, the distillation step comprising distilling a mixture comprising the first reaction mixture to produce a separated portion of the distillation mixture comprising a purified form of the compound of Formula (I) as compared to the first reaction mixture; and
(iii) contacting the separated portion with a nucleophile to produce a compound of formula (IV) wherein R¹ is selected from furyl, phenyl, C₁-C₁₀ alkyl, C₂-C₁₀ alkyl comprising at least one double bond or triplet bond, C₁-C₁₀ alkyl-OH, C₂-C₁₀alkyl-OH comprising at least one double bond or one triplet bond, and —CH₂-C≡C-Si-(R³)₃ wherein each R³ is selected independently from C₁-C₅ alkyl.

8. The method of claim 7 wherein the chloroformate is a compound of the Formula (VI), 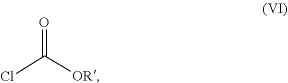
wherein R′ is selected from C₁-C₆ alkyl groups which may be optionally substituted, such that the carbamate has the formula Q-C(═O)OR′.

9. The method of claim 7 wherein the acid chloride is a compound of Formula (VII), 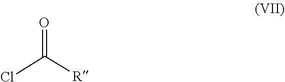
wherein R″ is selected from selected from selected from Cl, —C(O)Cl, phenyl wherein the phenyl ring is optionally substituted with one group selected from methyl, methoxy, halogen, C₁-C₁₀ alkyl and —C(O)Cl, bicyclic aromatic systems and tricyclic aromatic systems, such that the amide has the formula Q-C(═O) ″.

10. The method of claim 7, wherein the nucleophile is an organometallic reagent, for example, an allyl magnesium halide or an alkynyllithium.

11. The method of claim 7, wherein the produced compound of Formula (IV) is a compound selected from 6,10-dimethylundeca-5,9-dien-1-yne (e.g. (E)-6,10-dimethylundeca-5,9-dien-1-yne) and 6,10-dimethylundeca-1,5,9-triene (e.g. (E)-6,10-dimethylundeca-1,5,9-triene).

12. A compound of Formula (I), 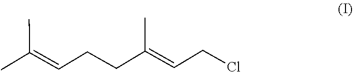
obtained by or obtainable by a method of any of claims 1 to 6.

13. A compound of Formula (IV), 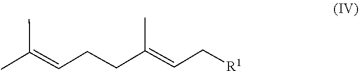
obtained or obtainable by a method of any one of claims 7 to 11.

14. A compound of claim 13, wherein the compound is selected from 6,10-dimethylundeca-5,9-dien-1-yne (e.g. (E)-6,10-dimethylundeca-5,9-dien-1-yne) and 6,10-dimethylundeca-1,5,9-triene (e.g. (E)-6,10-dimethylundeca-1,5,9-triene).
